(19) United States
(12) Patent Application Publication
(10) Pub. No.: US 2011/0137343 A1
(43) Pub. Date: Jun. 9, 2011

(54) PORTABLE SPINAL DISC DECOMPRESSION DEVICE

(75) Inventor: David M. Vitko, Columbiana, OH (US)

(73) Assignee: Spinal Innovations, LLC

(21) Appl. No.: 12/963,304

(22) Filed: Dec. 8, 2010

Related U.S. Application Data
(60) Provisional application No. 61/267,793, filed on Dec. 8, 2009.

(51) Int. Cl. A61F 5/00

(52) U.S. Cl. 606/240

(57) ABSTRACT

A portable spinal disc decompression device includes a collapsible base unit, a traction mechanism, a series of patient support members, and a controller. The base unit includes upper and lower base members. The traction mechanism includes first and second pluralities of linear bearings slidably connected to the upper and lower base members (respectively), and a linear actuator attached to the base unit. The patient support members are connected to the base unit and include a cervical support member, a thoracic support carriage, a pelvic support carriage, and first and second leg support members. The patient support members include a plurality of locking mechanisms that allow selective linear movement of the thoracic and pelvic support carriages. The controller is in electrical communication with the linear actuator and provides a signal to the linear actuator to cause a force to be applied to the thoracic and/or pelvic support carriages.

### RELATED APPLICATION

This application claims priority from U.S. Provisional Patent Application Ser. No. 61/267,793, filed Dec. 8, 2009, the subject matter of which is incorporated hereby incorporated by reference in its entirety.

### TECHNICAL FIELD

The present invention relates generally to a device for applying tractive forces to a subject, and more particularly to a portable device for selectively applying cervical, thoracic, and/or pelvic traction to treat back ailments in a subject.

### BACKGROUND OF THE INVENTION

Traction, also referred to as spinal decompression, is widely used to relieve pressure on inflamed or enlarged nerves. While traction is applicable to any part of the body, cervical and lumbar or spinal traction are the most common. When correctly performed, spinal traction can cause distraction or separation of the vertebral bodies, a combination of distraction and gliding of the facet joints, tensing of the ligamentous structures of the spinal segment, widening of the intervertebral foramen, straightening of spinal curvature, and stretching of the spinal musculature. Depending on the injury being treated, the traction component of physical therapy may require multiple sessions per week for a prolonged period of time.

Attempts to create a sufficiently low-cost, portable traction device for home use have thus far produced unsatisfactory results. A number of portable traction devices utilize pneumatic or hydraulic cylinders to create the traction force. Hydraulic cylinders have the disadvantage of the weight of the hydraulic fluid. Pneumatic cylinders with low pressure inputs typically cannot maintain an adequate traction force for a sufficient period of time to be effective in a traction device. In an attempt to overcome this deficiency, some of these devices utilize an automatic pumping device triggered by a pressure sensing device to supply additional compressed air so that a constant level of traction force is maintained. These pump and sensor configurations add cost, weight, and complexity to the traction device.

### SUMMARY OF THE INVENTION

According to one aspect of the present invention, a portable spinal disc decompression device for use in a home setting includes a collapsible base unit, a traction mechanism, a series of patient support members, and a controller. The base unit includes upper and lower base members, each of which include oppositely disposed first and second surfaces. The traction mechanism is securely connected to the first surface of each of the upper and lower base members. The traction mechanism includes a first plurality of linear bearings that is slidably connected to the upper base member, a second plurality of linear bearings that is slidably connected to the lower base member, and a linear actuator attached to the base unit. The series of patient support members are connected to the base unit and include a cervical support member that is connected to the upper base member, a thoracic support carriage that is slidably mounted to the first plurality of linear bearings, a pelvic support carriage that is slidably mounted to the second plurality of linear bearings, and first and second leg support members that are connected to the lower base member. The patient support members include a plurality of locking mechanisms that allow selective linear movement of the thoracic and pelvic support carriages. The controller is in electrical communication with the linear actuator. The controller is for communicating a signal to the linear actuator to cause the linear actuator to apply a force to at least one of the thoracic and pelvic support carriages.

According to another aspect of the present invention, a portable spinal disc decompression device for use in a home setting includes a collapsible base unit, a traction mechanism, a series of patient support members, and a controller. The base unit includes upper and lower base members, each of which include oppositely disposed first and second surfaces. The traction mechanism is securely connected to the first surface of each of the upper and lower base members. The traction mechanism includes a first plurality of linear bearings that is slidably connected to the upper base member, a second plurality of linear bearings that is slidably connected to the lower base member, and a linear actuator attached to the base unit. The series of patient support members are connected to the base unit and include a cervical support member that is connected to the upper base member, a thoracic support carriage that is slidably mounted to the first plurality of linear bearings, a pelvic support carriage that is slidably mounted to the second plurality of linear bearings, and first and second leg support members that are connected to the lower base member. Each of the first and second leg support members comprise upper and lower leg pads respectively mounted upon upper and lower leg plates. The upper and lower leg plates being hingedly connected to one another. Each of the upper leg plates being hingedly connected to the pelvic support carriage and each of the lower leg plates being connected to a lift mechanism for selectively elevating the leg support members relative to the lower base member. The controller is in electrical communication with the linear actuator. The controller is for communicating a signal to the linear actuator to cause the linear actuator to apply a force to at least one of the thoracic and pelvic support carriages.

According to another aspect of the present invention, a method is provided for spinal disc decompression in the home of a patient. One step of the method includes providing a portable spinal disc decompression device. The spinal disc decompression device includes a collapsible base unit, a traction mechanism, a series of patient support members, and a controller. The base unit includes upper and lower base members, each of which include oppositely disposed first and second surfaces. The traction mechanism is securely connected to the first surface of each of the upper and lower base members. The traction mechanism includes a first plurality of linear bearings that is slidably connected to the upper base member, a second plurality of linear bearings that is slidably connected to the lower base member, and a linear actuator attached to the base unit. The series of patient support members are connected to the base unit and include a cervical support member that is connected to the upper base member, a thoracic support carriage that is slidably mounted to the first plurality of linear bearings, and first and second leg support members that are connected to the lower base member. The series of patient support members includes a plurality of locking mechanisms. The controller is in electrical communication with the linear actuator. After providing the spinal disc decompression device, the patient is situated on the device so that the head, chest, waist, and legs of the patient respectively engage the cervical support member, the thoracic support carriage, the pelvic support carriage, and the first and second leg support members. At least one of the locking mechanisms is then selectively engaged to lock at least one of the thoracic or pelvic support carriages in place. Next, at least one of the first or second leg support members can be optionally elevated. The controller is then operated so that traction is applied to at least one of a cervical, thoracic, or thoracic/lumbar region of the patient.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a perspective view showing a top side of a spinal disc decompression device constructed in accordance with one aspect of the present invention;

FIG. 2A is a perspective view showing cervical, thoracic, and pelvic plates of the spinal disc decompression device in FIG. 1;

FIG. 2B is a perspective view showing a bottom side of the spinal disc decompression device in FIG. 2A;

FIG. 3A is a perspective view showing a base unit in an assembled configuration and being constructed in accordance with another aspect of the present invention;

FIG. 3B is a perspective view showing the base unit in FIG. 3A in a disassembled configuration;

FIG. 3C is a perspective view showing an alternative construction of the base unit in FIG. 3A in an assembled configuration;

FIG. 3D is a perspective view showing the base unit in FIG. 3C in a disassembled configuration;

FIG. 4A is a perspective view showing the base unit in FIG. 3A including an extension member;

FIG. 4B is a perspective view showing the base unit and extension member in FIG. 4A in an assembled configuration;

FIG. 5A is a perspective view showing a traction mechanism of the spinal disc decompression device in FIG. 1;

FIG. 5B is a magnified perspective view showing a portion of the traction mechanism in FIG. 5A disposed on an upper base member of the base unit;

FIG. 5C is a magnified perspective view showing another portion of the traction mechanism in FIG. 5A disposed on a lower base member of the base unit;

FIG. 6 is a perspective view showing an alternative configuration of the spinal disc decompression device in FIG. 1;

FIG. 7A is a perspective view showing a spinal disc decompression device with motorized first and second leg support members;

FIG. 7B is a magnified perspective view of the first and second leg support members in FIG. 7A;

FIG. 8A is a plan view showing a spinal disc decompression device with non-motorized first and second leg support members;

FIG. 8B is a side view of the first and second leg support members in FIG. 8A;

FIG. 9 is a perspective view of a hand-held controller for operating a spinal disc decompression device;

FIG. 10 is a process flow diagram showing a method for spinal decompression in the home of a patient according to another aspect of the present invention;

FIG. 11 is a perspective view showing a patient secured to the spinal disc decompression device in FIG. 1;

FIG. 12 is a perspective view showing the patient in FIG. 11 undergoing lumbar traction; and

FIG. 13 is a perspective view showing the patient in FIG. 11 undergoing cervical traction.

### DETAILED DESCRIPTION

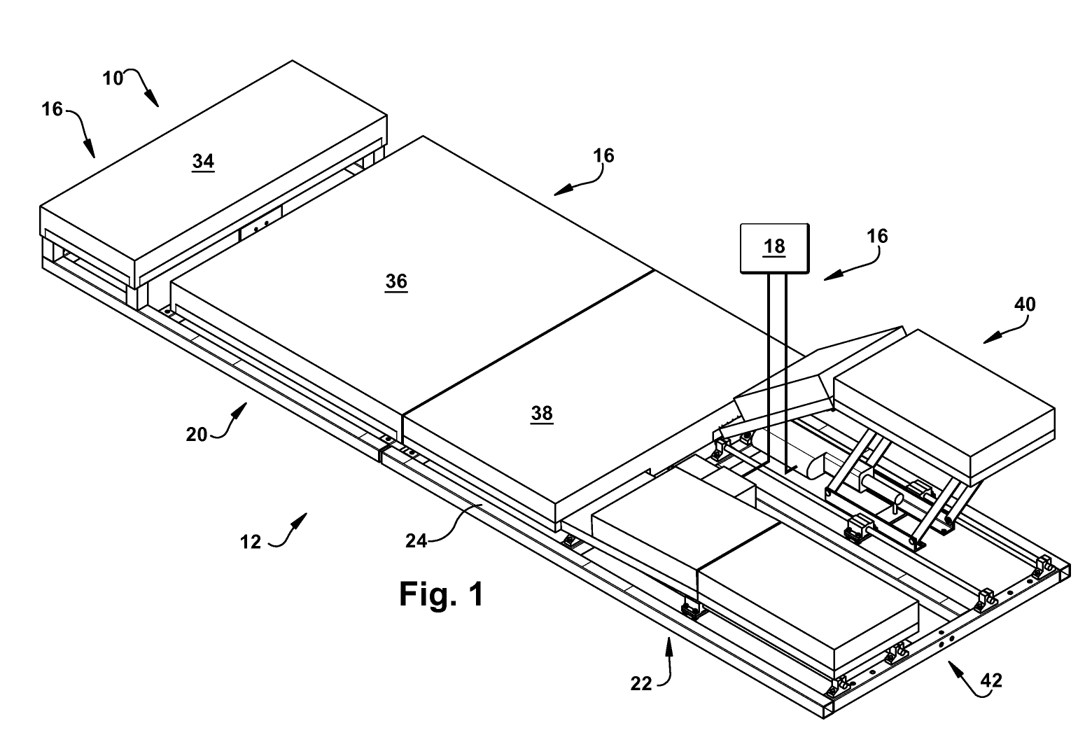

The present invention relates generally to a device for applying tractive forces to a subject, and more particularly to a portable device for selectively applying cervical, thoracic, and/or pelvic traction to treat back ailments in a subject. As representative of one aspect of the present invention, FIG. 1 illustrates a portable home spinal disc decompression device 10 for treating a variety of back ailments, such as disc-related, facet joint-related, degenerative disc-related, injury-related, and deconditioning-related back and neck pain. The present invention is based, at least in part, on the discovery that intermittent spinal disc decompression at lower poundages can be applied over a relatively long period of time (e.g., during nighttime) to substantially decrease healing time for back and leg pain. Based on this discovery, the present invention provides a lightweight, portable spinal disc decompression device 10 that can be used in a home setting to ameliorate a variety of back ailments.

Unlike conventional spinal traction devices, which are cumbersome and apply constant traction at high poundages for short durations of time, the present invention advantageously provides a spinal disc decompression device 10 that: (1) is portable and lightweight to facilitate transportation; (2) is capable of providing independent lumbar or cervical traction, as well as full spine traction; (3) is safe for home usage with integrated poundage limiters and emergency stop systems; (4) is user friendly as there are no ropes, pulleys, or problematic harnesses to deal with; (5) represents a departure from costly and inconvenient clinic-based spinal decompression treatments; (6) offers significantly longer treatment times, nighttime “treatment-while-you-sleep”, as well as increased treatment application times; and (7) includes software that is easily programmable and/or pre-programmed by medical practitioners (e.g., medical doctors, physical therapists, chiropractors, etc).

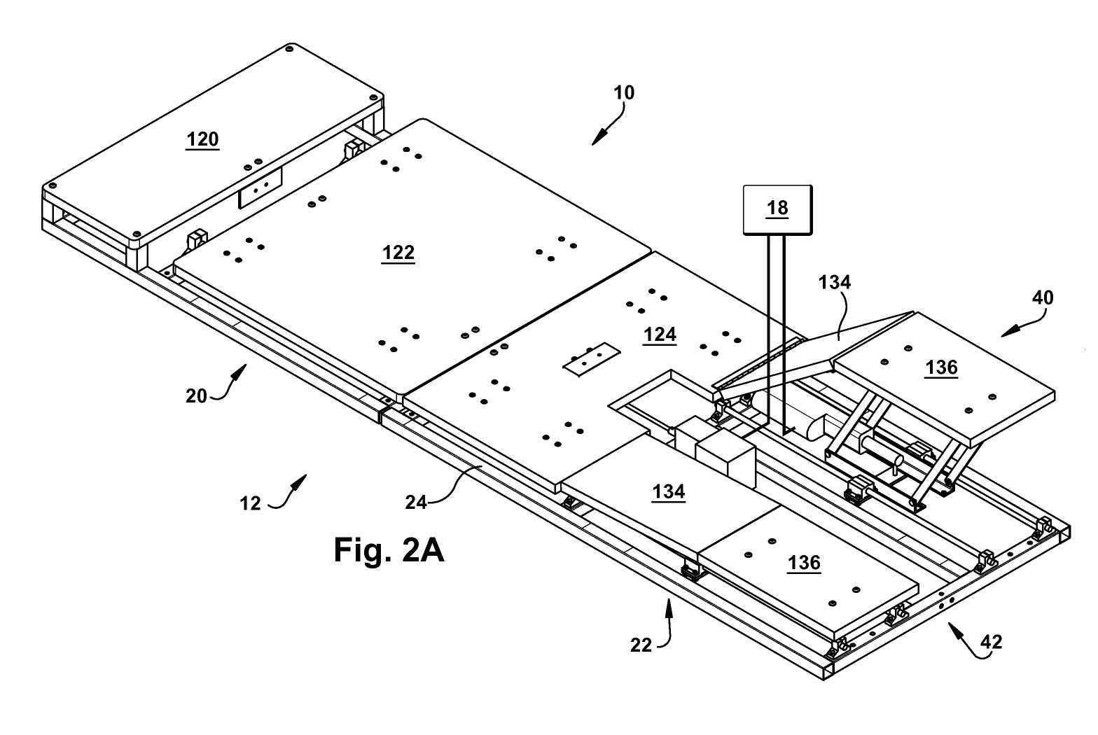

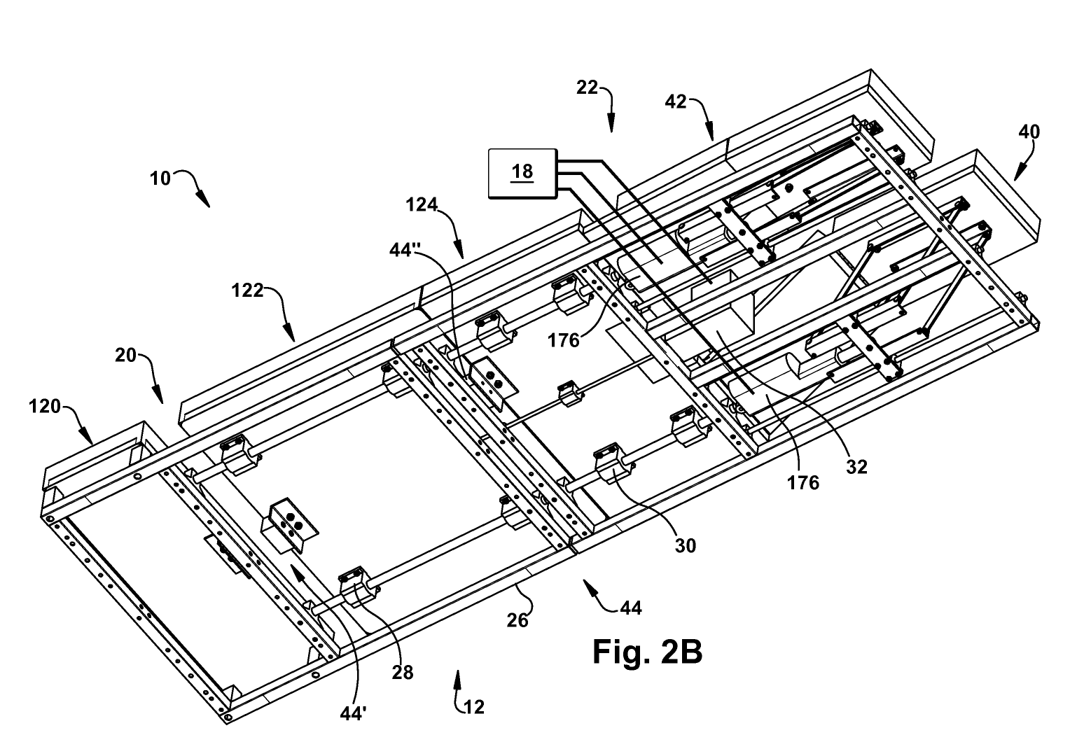

One aspect of the present invention includes a portable spinal disc decompression device 10 for use in a home setting. As shown in FIG. 1 and FIGS. 2A-B, the spinal disc decompression device 10 generally comprises a collapsible base unit 12, a traction mechanism 14, a series of patient support members 16, and a hand-held controller 18. The base unit 12 includes upper and lower base members 20 and 22, each of which include oppositely disposed first and second surfaces 24 and 26. The traction mechanism 14 is securely connected to the first surface 24 of each of the upper and lower base members 20 and 22. The traction mechanism 14 includes a first plurality of linear bearings 28 that is slidably connected to the upper base member 20, a second plurality of linear bearings 30 that is slidably connected to the lower base member 22, and a linear actuator 32 attached to the base unit 12. The series of patient support members 16 are connected to the base unit 12 and include a cervical support member 34 that is connected to the upper base member 20, a thoracic support carriage 36 that is slidably mounted to the first plurality of linear bearings 28, a pelvic support carriage 38 that is slidably mounted to the second plurality of liner bearings 30, and first and second leg support members 40 and 42 that are connected to the lower base member 22. The patient support members 16 include a plurality of locking mechanisms 44 that allow selective linear movement of the thoracic and pelvic support carriages 36 and 38. The controller 18 is in electrical communication with the linear actuator 32 and is for communicating a signal to the linear actuator to cause the linear actuator to apply a force to at least one of the thoracic and pelvic support carriages 36 and 38.

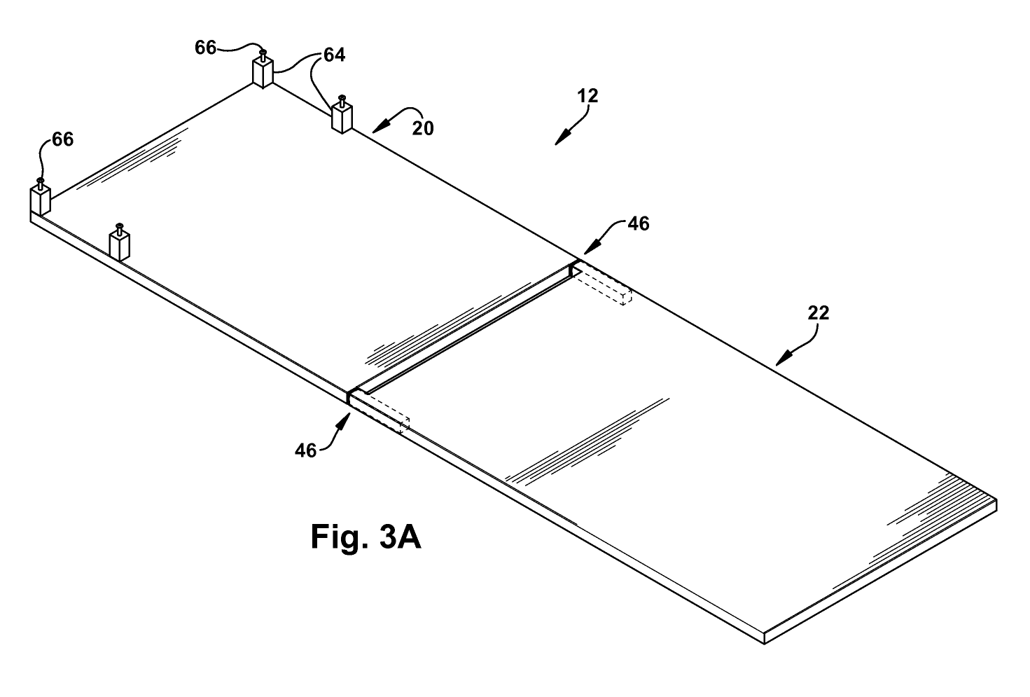

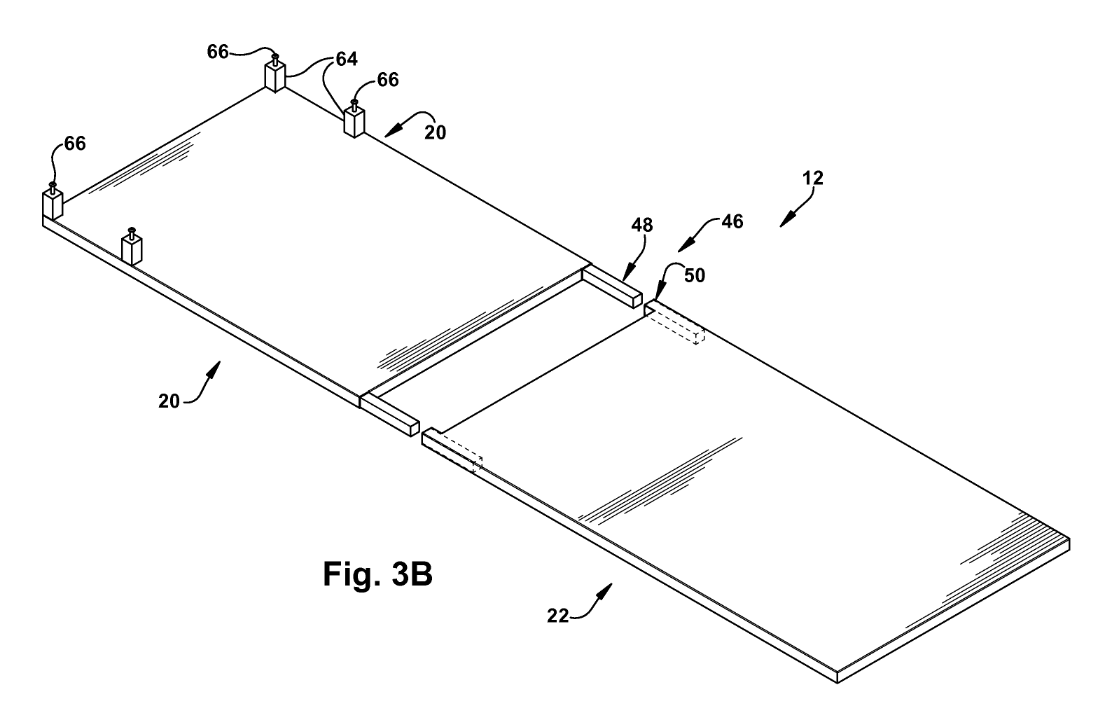

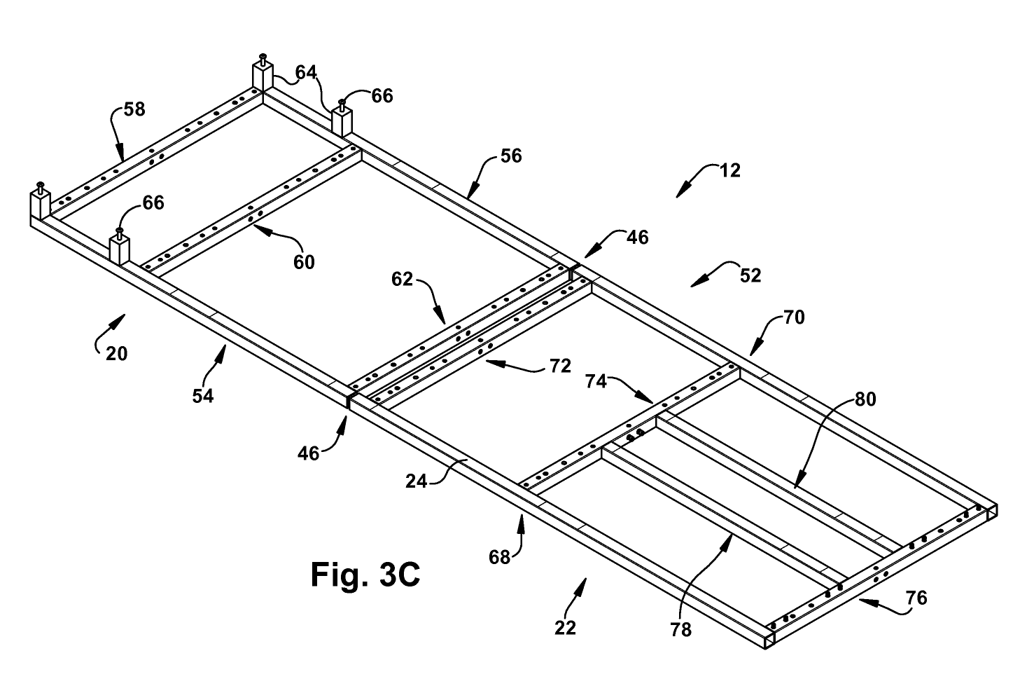

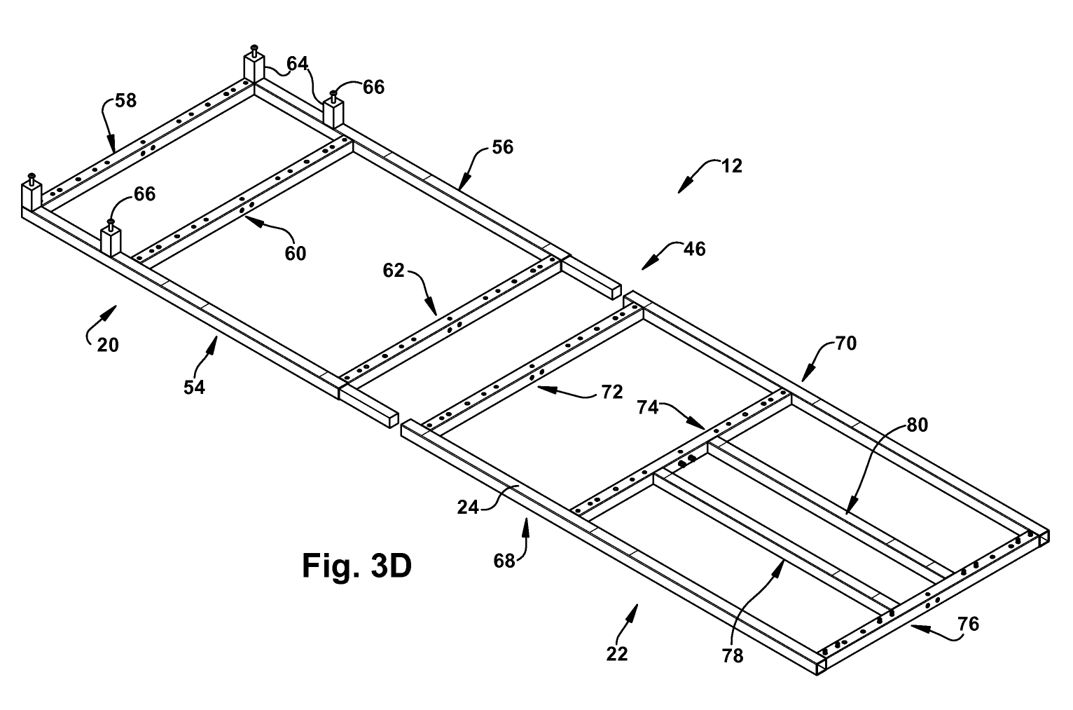

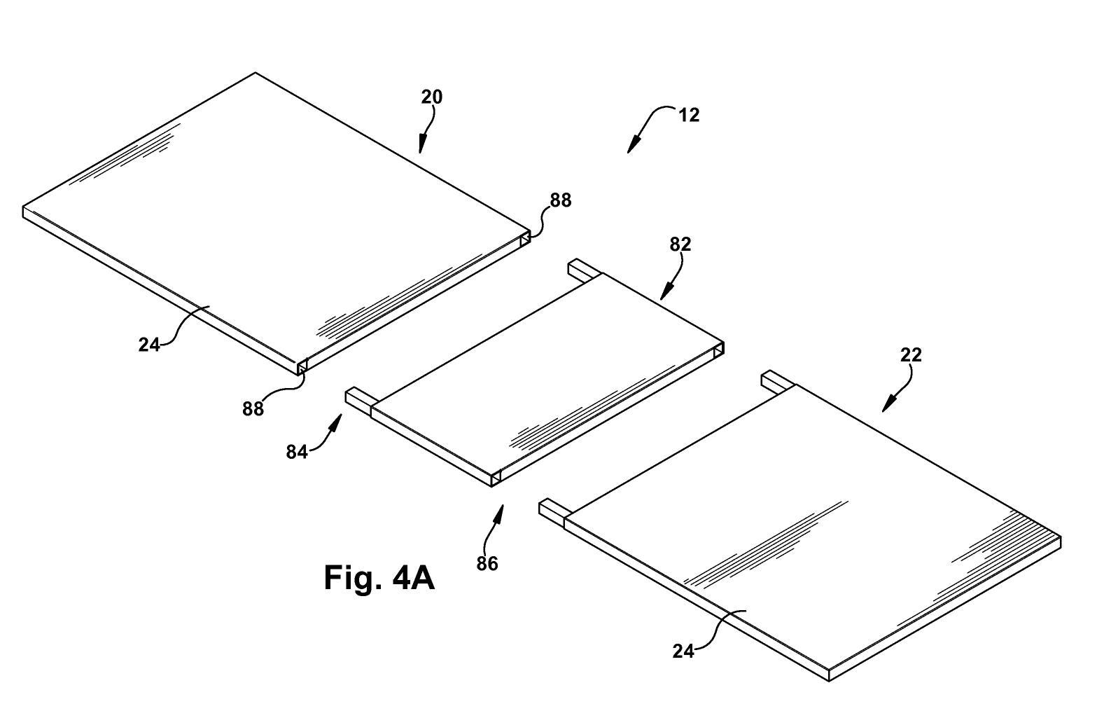

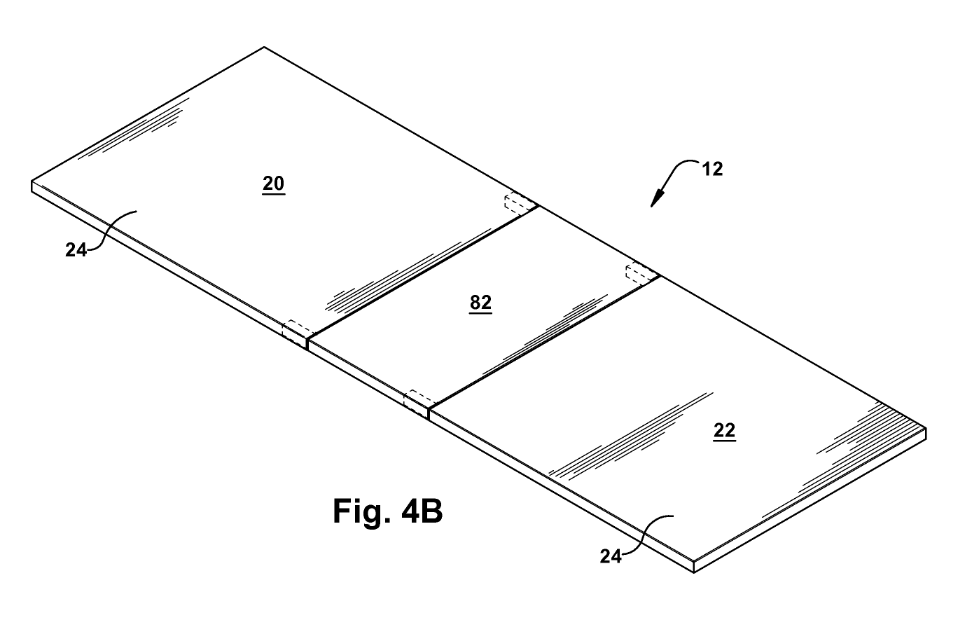

As shown in FIGS. 3A-4B, the collapsible base unit 12 includes an upper base member 20 and lower base member 22 upon which the functional components of the spinal disc decompression device 10 are supported. All or only a portion of each of the upper and lower base members 20 and 22 can be made of a metal, metal alloy and/or plastic. Each of the upper and lower base members 20 and 22 includes oppositely disposed first and second surfaces 24 and 26. The base unit 12 has an elongated, generally rectangular shape for accommodating and supporting a patient. As described in more detail below, the actual dimensions of the base unit 12 can vary depending upon the size of the patient.

The base unit 12 includes a series of selectively releasable attachment mechanisms 46 for easily attaching and disconnecting the upper and lower base members 20 and 22. Each of the releasable attachment mechanisms 46 generally comprises male and female connecting members 48 and 50 that are formed as part of the upper and lower base members 20 and 22, respectively, and are capable of being snap-fit together. Each of the releasable attachment mechanisms 46 can additionally or optionally include at least one adjustable pin (not shown) that can be selectively engaged to securely connect or disconnect the upper and lower base members 20 and 22.

In one example of the present invention, the base unit 12 can be made entirely of plastic (FIGS. 3A-B). In this case, the upper and lower base members 20 and 22 can be formed using plastic sheets, tubes, and/or or corrugated plastic sheets. A known plastic molding technique, such as blow molding can be used to form the base unit 12. It will be appreciated that all or only a portion of each of the upper and lower base members 20 and 22 can be hollow to make the spinal disc decompression device 10 as lightweight as possible (e.g., about 50 lbs.). As shown in FIG. 3B, each of the female connecting members 50 of the releasable attachment mechanisms 46 can comprise a receptacle portion (not shown in detail) that is disposed within the lower base member 22.

In another example of the present invention, each of the upper and lower base members 20 and 22 of the base unit 12 can comprise a series tube-like members 52 (FIGS. 3C-D). One or all of the tube-like members 52 can be made of a lightweight and rigid material, such as a metal, metal alloy or plastic. As shown in FIG. 3C, the upper base member 20 comprises oppositely disposed first and second longitudinal cervical tube members 54 and 56 and first, second, and third cross tube members 58, 60, and 62 that extend between the first and second longitudinal cervical tube members. As explained in more detail below, the upper base member 20 additionally includes four cervical plate spacers 64 for supporting the cervical support member 34. Each of the cervical plate spacers 64 are about equally-sized and securely connected to the first and second longitudinal cervical tube members 54 and 56. Each of the cervical plate spacers 64 can be made of the same or different material(s) as the first and second longitudinal cervical tube members 54 and 56, and be adapted to mate with a threaded screw 66 (e.g., a socket button head cap screw).

The lower base member 22 comprises oppositely disposed first and second longitudinal pelvic tube members 68 and 70. Fourth, fifth, and sixth cross tube members 72, 74, and 76 are securely disposed between the first and second longitudinal pelvic tube members 68 and 70. As shown in FIG. 3C, oppositely disposed first and second longitudinal pelvic short tube members 78 and 80 extend between the fifth and sixth cross tube members 74 and 76.

As mentioned above, the dimensions of the base unit 12 can vary depending upon the size of the patient. To accommodate taller patients, for example, at least one extension member 82 (FIGS. 4A-B) can be disposed between the upper and lower base members 20 and 22. The extension member 82 can be made of the same or different material(s) as the upper and lower base members 20 and 22, and have a width and height that are about equal to the height and width of each of the upper and lower base members. The length of the extension member 82 can be varied depending upon the height of the subject. The extension member 82 can include oppositely disposed first and second mating end portions 84 and 86 that are respectively adapted to securely mate with the upper and lower base members 20 and 22, respectively. As shown in FIGS. 4A-B, the base unit 12 can be lengthened to accommodate a taller patient by inserting the first mating end portion 84 into a corresponding female portion 88 of the upper base member 20, and mating the lower base member 22 with the second mating end portion 86 of the extension member 82. The extension member 82 can be securely mated with the upper and lower base members 20 and 22 via a snap-fit, friction-fit, or other suitable connecting mechanism.

In another aspect of the present invention, the spinal disc decompression device 10 includes a traction mechanism 14 securely connected to the first surface 24 of each of the upper and lower base members 20 and 22. The traction mechanism 14 is capable of providing a force to at least one of the thoracic support carriage 36 or the pelvic support carriage 38, which allows for lower back traction (spinal disc decompression) separately, or in combination with, thoracic and cervical traction. As described in more detail below, movement of the thoracic support carriage 36 and/or the pelvic support carriage 38 is facilitated by the linear actuator 32, which has sufficient power and durability to develop traction forces to positively influence the health of spinal discs when properly applied to the patient.

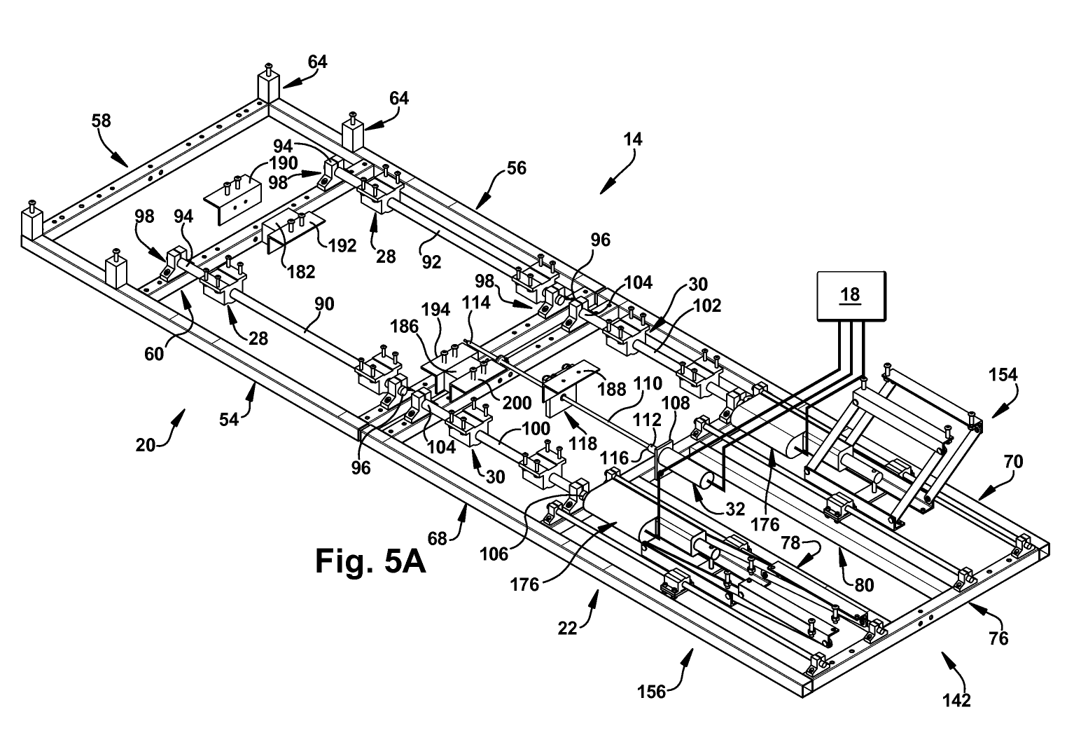

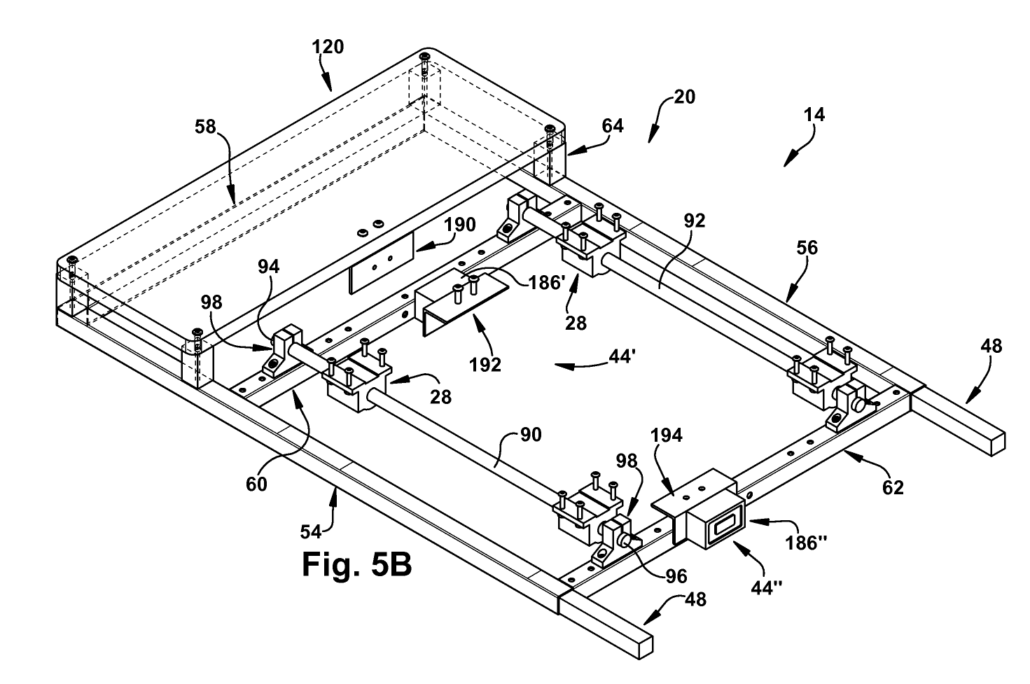

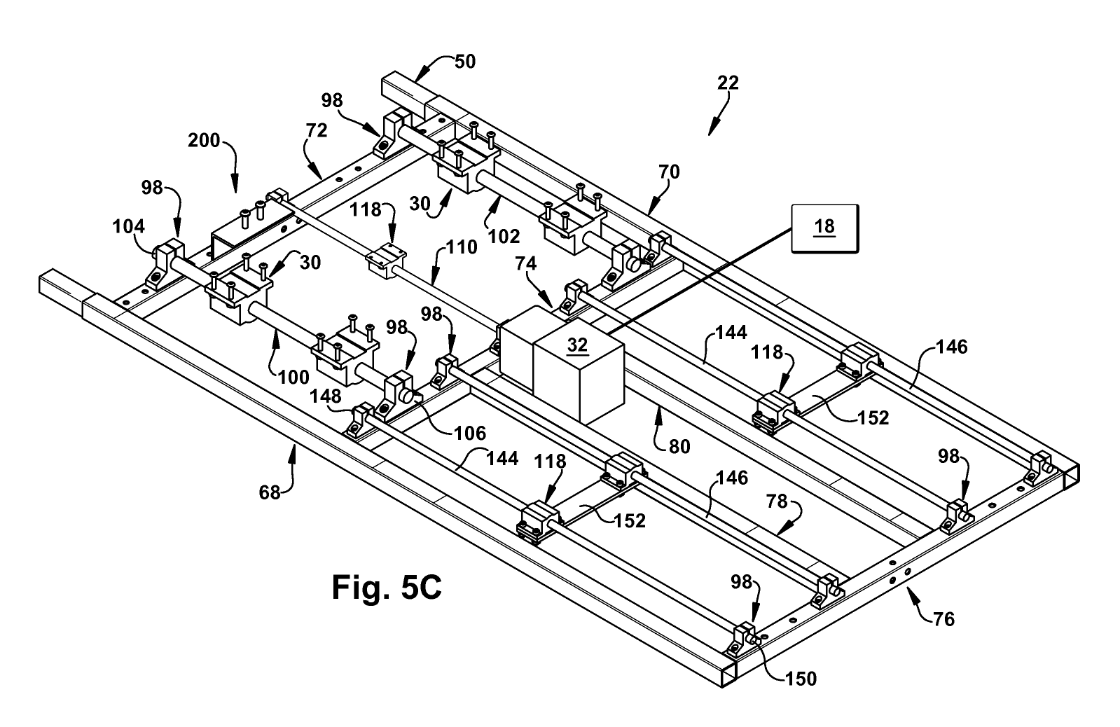

As shown in FIGS. 5A-B, a portion of the traction mechanism 14 comprises a first plurality of linear bearings 28 that is slidably connected to the upper base member 20. For example, each of the first plurality of linear bearings 28 (e.g., closed pillow blocks) is slidably mounted to oppositely disposed first and second shaft members 90 and 92. The first plurality of linear bearings 28 allows the thoracic support carriage 36 to be selectively moved in either the foot ward or head ward direction along the upper base member 20. The first plurality of linear bearings 28 permits the thoracic support carriage 36 to be securely mounted thereto via a plurality of screws 66 (e.g., socket button head cap screws). Each of the first and second shaft members 90 and 92 includes first and second ends 94 and 96, each of which is respectively attached to the second and third cross tube members 60 and 62 via a series of shaft support blocks 98.

A portion of the traction mechanism 14 additionally comprises a second plurality of linear bearings 30 that is slidably connected to the lower base member 22. As shown in FIG. 5C, each of the second plurality of linear bearings 30 (e.g., closed pillow blocks) is slidably mounted to oppositely disposed third and fourth shaft members 100 and 102. The second plurality of linear bearings 30 allows the pelvic support carriage 38 to be selectively moved in either the foot ward or head ward direction along the lower base member 22. The second plurality of linear bearings 30 permits the pelvic support carriage 38 to be securely mounted thereto via a plurality of screws 66 (e.g., socket button head cap screws). Each of the third and fourth shaft members 100 and 102 includes first and second ends 104 and 106, each of which is respectively attached to the fourth and fifth cross tube members 72 and 74 via a series of shaft support blocks 98.

As noted above, the traction mechanism 14 additionally includes a linear actuator 32 that is capable of applying a force to at least one of the thoracic support carriage 36 or the pelvic support carriage 38. The linear actuator 32 can generally include a motor and a gear reducer (or other type of compact drive device) that is securely mounted to the base unit 12. As shown in FIG. 5A, for example, the linear actuator 32 is securely mounted to the fifth cross tube member 74 via a motor mount 108. Examples of linear actuators are known in the art. The linear actuator 32 is operably connected to a drive rod 110 via a motor coupler 112. The drive rod 110 includes oppositely disposed first and second ends 114 and 116, each of which is respectively connected to a shaft support block 98 that is securely connected to the fourth and fifth cross tube members 72 and 74. A closed pillow block 118 is slidably mounted to the drive rod 110 to support the pelvic support carriage 38.

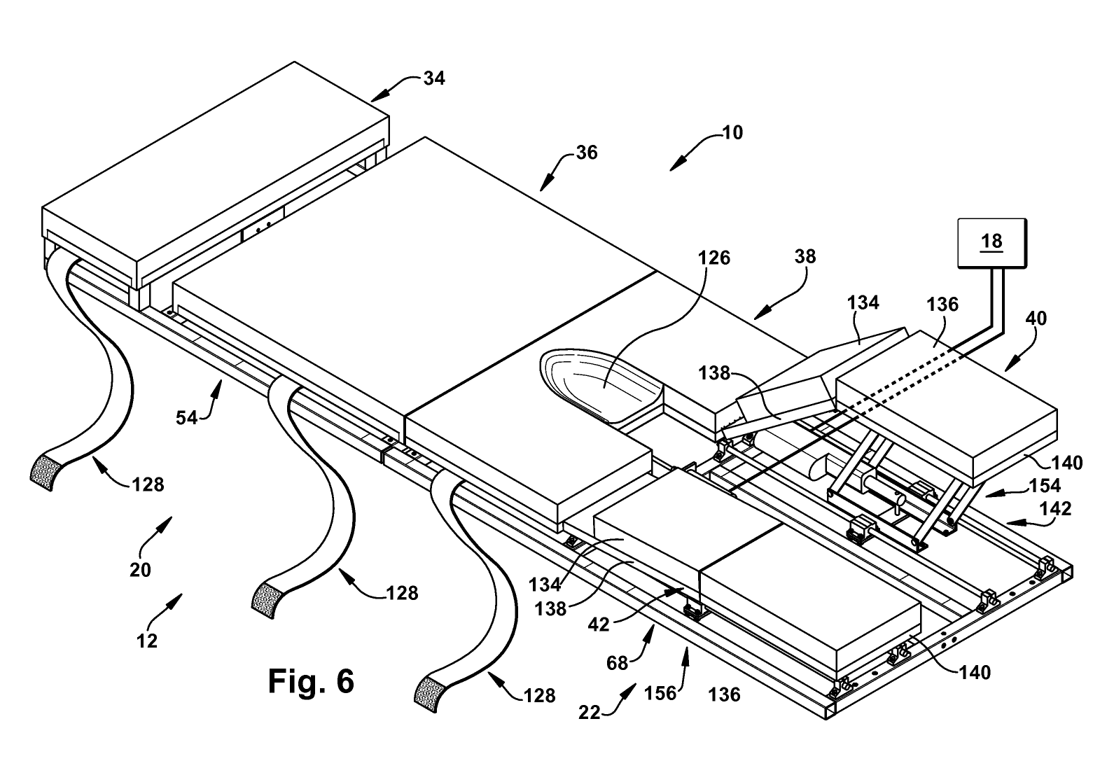

In another aspect of the present invention, the spinal disc decompression device 10 includes a series of patient support members 16 (FIG. 6) connected to the base unit 12. As shown in FIG. 6, the series of patient support members 16 includes a cervical support member 34 that is connected to the upper base member 20 via cervical plate 120, a thoracic support carriage 36 that is slidably mounted to the first plurality of linear bearings 28 via a thoracic plate 122, and a pelvic support carriage 38 that is slidably mounted to the second plurality of linear bearings 30 via a pelvic plate 124. The patient support members 16 are generally comprised of a pad or pillow-like material that is capable of comfortably supporting the patient during use of the spinal disc decompression device 10 (e.g., for extended periods of time). Each of the patient support members 16 can have a desired thickness and be made of any one or combination of materials, such as rubber, cloth, etc. A patient-contacting surface of each of the patient support members 16 includes a non-slip surface, which takes advantage of the naturally-occurring friction forces between the patient's body and the surface of each of the patient support members during operation of the spinal disc decompression device 10.

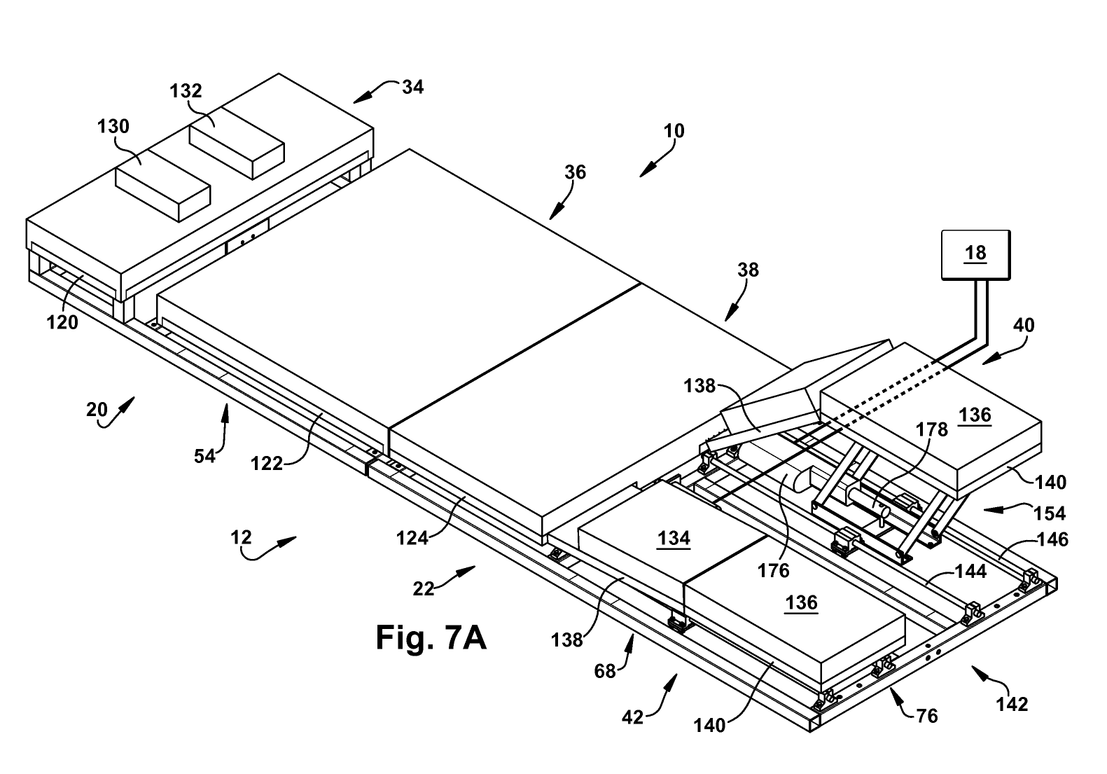

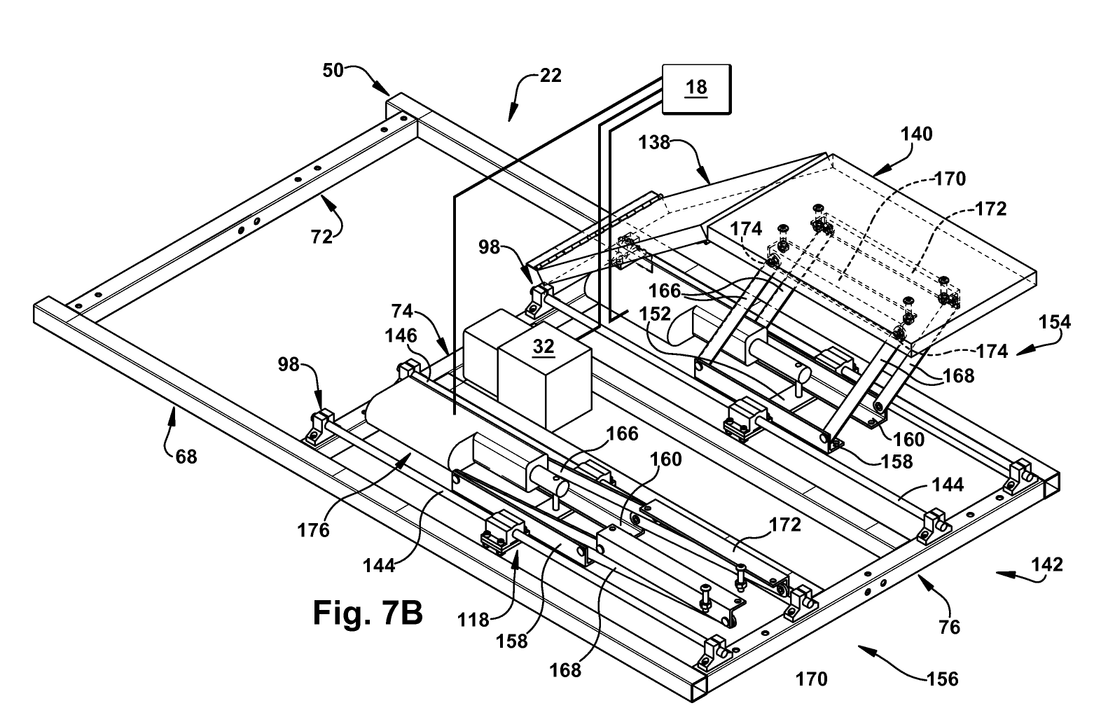

One or more of the patient support members 16 can include a recessed portion 126 that is adapted to receive an ice and/or gel pack (not shown) during treatment. As shown in FIG. 6, the pelvic support carriage 38 can include a recessed portion 126 located at the midline of the pelvic support carriage. Additionally, one or more of the patient support members 16 can include a strap or belt member 128 for mechanically securing the patient to the spinal disc decompression device 10. The belt or strap member 128 stabilizes the patient's body to each patient support member 16 during operation of the spinal disc decompression device 10. The strap or belt members 128 can be pulled across the patient's body and affixed by simple hook-and-eye closures (not shown). It will be appreciated that the patient support members 16 can include other features to increase patient comfort, such as first and second facial pads 130 and 132 (FIG. 7A) that are disposed on the cervical support member 34. The first and second facial pads 130 and 132 can support the patient's head when using the spinal disc decompression device 10 in a face-down position. Additionally, it will be appreciated that the thoracic support carriage 36 and/or the pelvic support carriage 38 may be capable of elevation to improve the comfort of patients, especially when lying prone on the spinal disc decompression device 10.

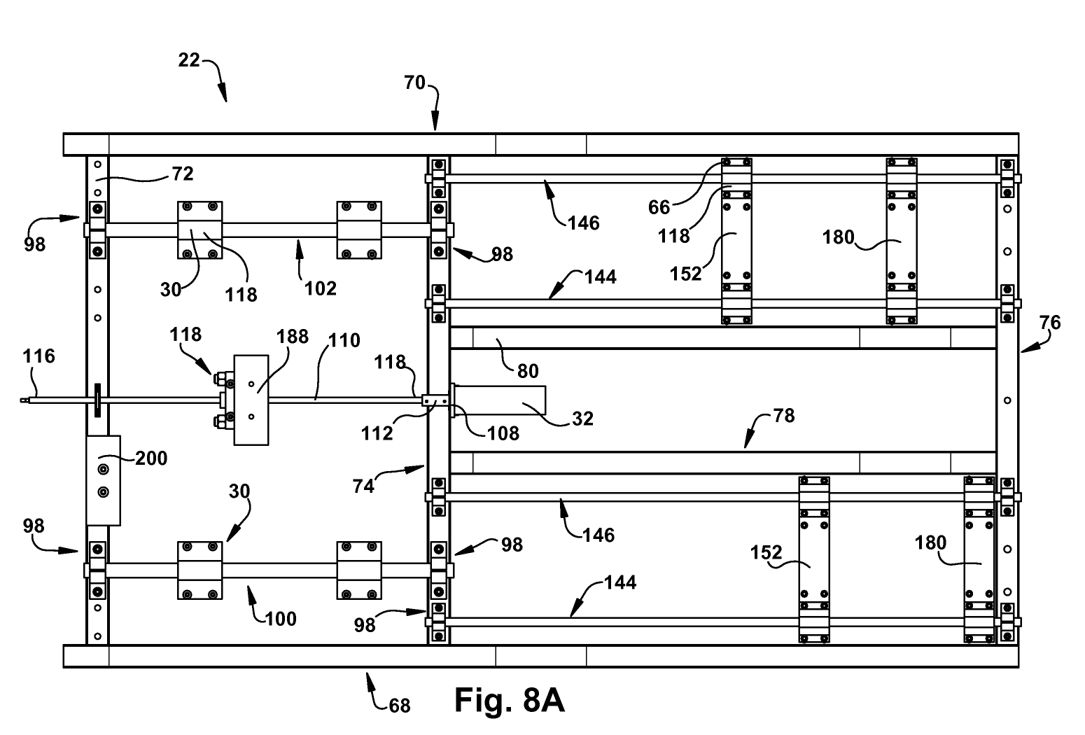

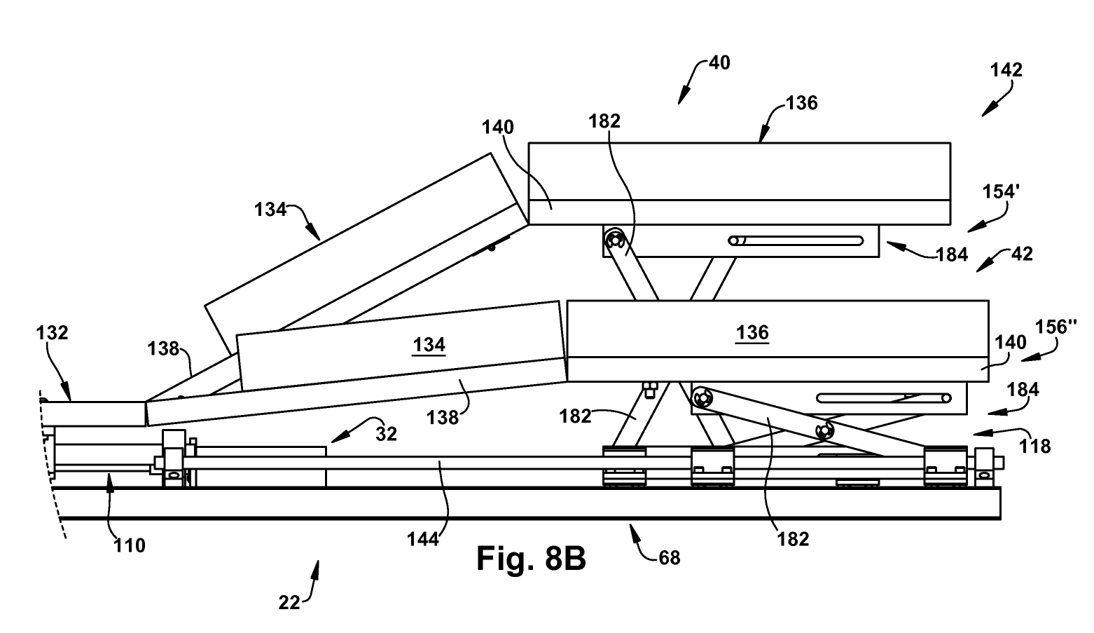

In another aspect of the present invention, the spinal disc decompression device 10 includes first and second leg support members 40 and 42 (FIGS. 7A-B) that are operably connected to the lower base member 22. As described in more detail below, the spinal disc decompression device 10 can include first and second leg support members 40 and 42 that are either motorized (FIGS. 7A-B) or non-motorized (FIGS. 8A-B). Both the motorized and non-motorized configurations can be used to treat sciatic pain in the left and/or right leg(s) of the patient via selective elevation of the first leg support member 40 and/or the second leg support member 42 (respectively).

Referring to FIGS. 7A-B, each of the first and second leg support members 40 and 42 comprises upper and lower leg pads 134 and 136 respectively mounted upon upper and lower leg plates 138 and 140. The upper and lower leg pads 134 and 136 can be similarly constructed as the patient support members 16; that is, each of upper and lower leg pads can generally be comprised of a pad or pillow-like material having a non-slip patient-contacting surface. The upper leg plate 138 of each of the first and second leg support members 40 and 42 is separately attached to the thoracic plate 122 via at least one piano hinge (not shown in detail). Additionally, the upper and lower leg plates 138 and 140 of each of the first and second leg support members 40 and 42 are attached to one another via a hinge (not shown in detail). As described in more detail below, the lower leg plate 140 of each of the first and second leg support members 40 and 42 is operably connected to a lift mechanism 142 for selectively elevating the first and second leg support members 40 and 42.

The lift mechanism 142 of each of the first and second leg support members 40 and 42 comprises first and second linkage bars 144 and 146 that extend longitudinally between the fifth and sixth cross tube members 74 and 76. Each of the first and second linkage bars 144 and 146 includes oppositely disposed first and second ends 148 and 150 that are respectively secured to the fifth and sixth cross tube members 74 and 76 via shaft support blocks 98. Additionally, each of the first and second linkage bars 144 and 146 includes a closed pillow block 118 and a first leg slide plate 152 that is securely connected to, and extends between, the closed pillow blocks.

Referring to the motorized configuration of the first and second leg support members 40 and 42 (FIGS. 7A-B), the lift mechanism 142 additionally includes first and second elevation assemblies 154 and 156 that operably join each of the lower leg plates 140 and the first leg slide plate 152. Each of the first and second elevation assemblies 154 and 156 comprises first and second clevis members 158 and 160 that each extend transverse to, and are securely connected with, the first leg slide plate 152. Each of the first and second clevis members 158 and 160 extend substantially parallel to the first and second linkage bars 144 and 146 and include oppositely disposed first and second ends 162 and 164. Slidably attached to the first and second ends 162 and 164 of each of the first and second clevis members 158 and 160 are third and fourth linkage bars 166 and 168. The third and fourth linkage bars 166 and 168 extend between the first and second clevis members 158 and 160 and third and fourth clevis members 170 and 172. The third and fourth linkage bars 166 and 168 are operably connected to the first, second, third, and fourth clevis members 158, 160, 170, and 172 by a series of rivets 174.

The lift mechanism 142 further includes at least one motor 176. As shown in FIGS. 7A-B, a motor 176 is securely mounted to the fifth cross tube member 74 and includes a drive rod 178 that extends from the motor to the first elevation assembly 154. Although not shown, it will be appreciated that a second motor can also be mounted to the fifth cross tube member 74 and include a drive rod that extends from the second motor to the second elevation assembly 156. The drive rod 178 is operably connected to a portion of the second clevis member 160 of the first elevation assembly 154. In operation, the controller 18 is selectively operated to activate the motor 176 and thereby elevate or depress the first leg support member 40. To elevate the first leg support member 40, for example, the motor 176 is activated so that a linear force is applied to the drive rod 178 in a head ward direction. Application of the linear force causes the first elevation assembly 154 to slide along the first and second linkage bars 144 and 146. As the first elevation assembly 154 is advanced along the first and second linkage bars 144 and 146, the upper leg plate 138 moves tangentially to the lower base member 22 and the lower leg plate 140 moves in a head ward direction while also remaining substantially parallel to the lower base member.

Referring to the non-motorized configuration of the first and second leg support members 40 and 42 (FIGS. 8A-B), the lift mechanism 142′ is similar to the lift mechanism 142 of the motorized configuration, except that the lift mechanism comprising the non-motorized configuration does not include a motor 176, and the first and second elevation assemblies 154 and 156 are constructed differently. For example, the lift mechanism 142′ includes a second leg slide plate 180 that is slidably connected to the first and second linkage bars 144 and 146 via a plurality of closed pillow blocks 118. Additionally, the first and second elevation assemblies 154′ and 156′ have a scissor lift or jack-type configuration comprising a series of cross bars 182 operably mounted to oppositely disposed clevis members 184. The first and second leg support members 40 and 42 can be manually adjusted (i.e., elevated or depressed) as needed by the patient or a medical practitioner.

In another aspect of the present invention, the spinal disc decompression device 10 includes a plurality of locking mechanisms 44 (FIGS. 5A-C) for allowing selective linear movement of the thoracic and pelvic support carriages 36 and 38. Each of the locking mechanisms 44 comprises an electromagnetic member 186 and a series of angle iron plates 188. A first angle iron plate 190 is securely attached to a second end (not indicated) of the cervical plate 120, second and third angle iron plates 192 and 194 are securely attached to first and second ends 196 and 198 of the thoracic plate 122, and a fourth angle iron plate 200 is securely attached to a first end 202 of the pelvic plate 124. First and second electromagnetic members 186′ and 186″ are securely connected to the second and third angle iron plates 192 and 194, respectively. It will be appreciated that the first and second electromagnetic members 186′ and 186″ can have a configuration other than the block-shaped configuration shown in FIG. 5A. Although described in more detail below, the locking mechanisms 44 generally function by magnetically mating the electromagnetic members 186 with an appropriate one of the angle iron plates 188 to fix the pelvic support carriage 38 and/or the thoracic support carriage 36 in place and thereby isolate one or more sections of the patient's spine (e.g., cervical, thoracic, lumbar) during application of a linear force by the linear actuator 32.

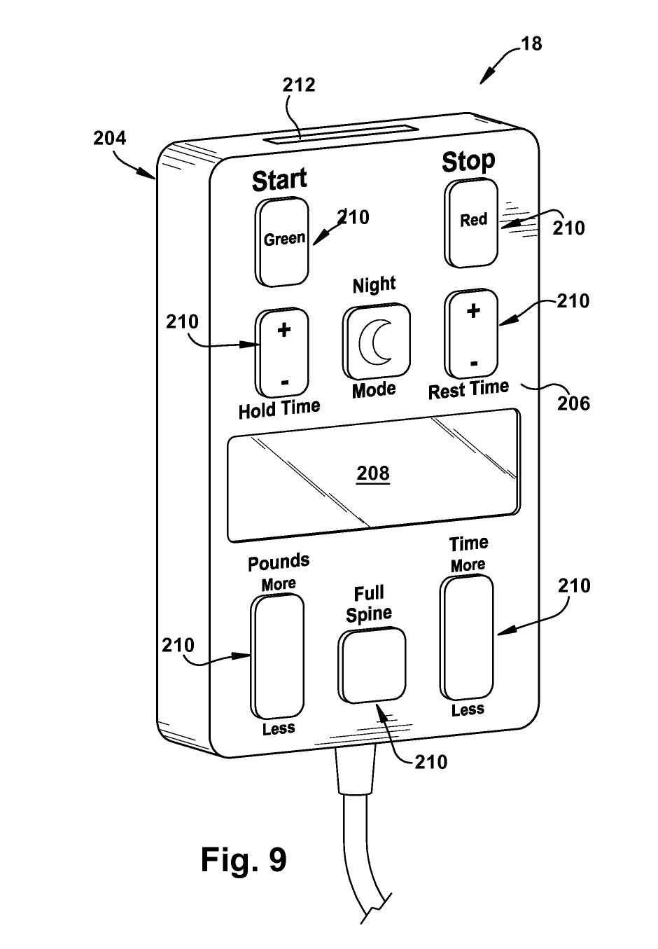

In another aspect of the present invention, the spinal disc decompression device 10 includes a hand-held controller 18 (FIG. 9) that is in electrical communication with the linear actuator 32 and/or the motor(s) 176 used to operate the first and second leg support members 40 and 42 (for the motorized configuration). Generally, the controller 18 allows a patient and/or medical practitioner to select numerous different options for treatment times, traction strength, hold times, rest times, relax times, and even variations in pull patterns. As described in more detail below, the controller 18 includes software that permits a patient and/or medical practitioner to manually input such treatment options or, alternatively, input pre-programmed treatment protocols that are specifically prescribed based on the patient's particular back ailment(s). Unlike conventional traction devices, the controller 18 of the present invention permits the application of intermittent traction at lower poundages and longer treatment times in a home setting within the acceptable relevant protocols for safe and effective traction treatment.

As noted above, the controller 18 is in electrical communication with the linear actuator 32 and/or the motor(s) 176 used to operate the first and second leg support members 40 and 42 (for the motorized configuration). For example, the controller 18 can be in electrical communication with the linear actuator 32 and/or the motor(s) 176 via a hard-wired or wireless arrangement. The controller 18 can be in direct electrical communication with the linear actuator 32 and/or motor(s) 176 or, alternatively, in indirect communication via an electronic circuit control panel (not shown) that is affixed to the spinal disc decompression device 10. The electronic circuit control panel can distribute power to the linear actuator 32 and/or motor(s) 176 from a power source (not shown). For example, the electronic circuit control panel can be electronically connected with a standard wall outlet or, alternatively, be powered by one or more batteries. In one example of the present invention, the power may be converted from AC power (e.g., from a wall outlet) to DC power (low voltage) via an on-board voltage converter (not shown) and in conjunction with the electronic circuit control panel.

The controller 18 generally comprises a housing 204, circuitry (not shown), and software. As shown in FIG. 9, the controller 18 includes a housing 204 that is ergonomically and aesthetically adapted for comfort and ease of use. The housing 204 has a generally rectangular shape defined by a front side 206 that is oppositely disposed from a back side (not shown). The housing 204 can be made from one or a combination of durable materials, such as metals, metal alloys, plastics (e.g., hardened plastics), and various other known polymers. One skilled in the art will recognize that the shape of the housing 204 described and shown herein should not be limiting. Although not shown in FIG. 9, it will be appreciated that the controller 18 can additionally include at least one power source that is operably connected to, and in electrical communication with, the housing 204 (e.g., the circuitry). For instance, the power source can be disposed within the housing 204 as a single life or rechargeable battery. Alternatively, the controller 18 can be powered by a DC or AC power source via the electronic circuit control panel.

The housing 204 also includes a user interface 208 that is operably connected to the front side 206 of the housing. The user interface 208 can generally include any type of two-dimensional or three-dimensional display screen, such as an LCD screen with a resolution capable of displaying information and/or permitting information exchange between a patient and/or medical practitioner and the controller 18. For example, the user interface 208 can permit the graphical and/or textual exchange of information between a patient and/or medical practitioner and the controller 18. The user interface 208 is sized to occupy a portion of the front side 206 of the controller 18. It will be appreciated that the user interface 208 can be smaller or larger than the one shown in FIG. 9, and that the user interface can have any other desired shape (e.g., circular, ovoid, etc). Additionally, it will be appreciated that the controller 18 can include more than one user interface 208. One example of the user interface 208 can include a graphical user interface (GUI), which allows a patient and/or medical practitioner to interact with the controller 18 in more ways than just typing or depressing buttons. For example, a GUI can offer graphical icons and visual indicators, as opposed to text-based interfaces, typed command labels, or text navigation to fully represent information to a patient and/or medical practitioner.

The controller 18 additionally includes circuitry for collecting, storing, and relaying treatment data. The ability of the controller 18 to store treatment data can be used to match treatment data to patient self-reported pain forms from the doctor's office to provide a “double-blind study effect”. Although not shown, the circuitry is in communication with one or more operational control buttons 210 that can be manipulated to control certain operations of the spinal disc decompression device 10, such as the start/stop time, hold time, rest time, amount of applied poundage, treatment time, day/night mode, and type of traction (e.g., full spine).

As used herein, the term “circuitry” can include electrical circuitry having at least one discrete electrical circuit, electrical circuitry having at least one integrated circuit, electrical circuitry having at least one application-specific integrated circuit, electrical circuitry forming a general purpose computing device configured by a computer program (e.g., a general purpose computer configured by a computer program that at least partially carries out processes described herein, or a microprocessor configured by a computer program that at least partially carries out processes described herein), electrical circuitry forming a memory device (e.g., forms of memory, such as random access, flash, read only, etc.), electrical circuitry forming a communications device (e.g., a modem, communications switch, optical-electrical equipment, etc.), and/or any non-electrical analog thereto, such as optical or other analogs. Those having skill in the art will recognize that the circuitry can be implemented in an analog fashion, a digital fashion, or some combination thereof.

Additionally, the controller 18 includes software for implementing a programmed and/or pre-programmed treatment protocol. The software can generally include one or more computer programs and related data that provide instructions to the circuitry. The software can comprise one or more known types of software, such as system software (e.g., an operating system), programming software (e.g., defining the syntax and semantics of various programs), and application software (e.g., end-user applications). Other examples of software can include firmware, device drivers, programming tools, and middleware. In one example of the present invention, the software can include a program that is capable of discerning between each type of programmed or pre-programmed traction protocol and automatically control (e.g., lock) appropriate patient support members to allow specific areas of the body to safely receive traction. Additionally or optionally, the software can include “default treatment programs” for lower back, cervical spine, or full spine treatments for simplicity.

It will be appreciated that the controller 18 can include one or more optional components. For example, the controller 18 can include a control interface (not shown), such as a keyboard that allows a patient or a third party (e.g., a medical practitioner) to directly enter data into the controller. As shown in FIG. 9, the controller 18 can also include one or more input/output (I/O) ports 212. An I/O port 212 can be used to transfer data into and/or out of the controller 18. For example, the I/O port 212 can comprise a slot for a memory card (not shown) that is preprogrammed (e.g., by the patient's doctor) with a specifically prescribed treatment protocol. Alternatively, the electronic circuit control panel can include a slot for the memory card. The memory card can be programmed with all the patient's treatment parameters so that the patient can simply plug the memory card into the controller 18 (or the electronic circuit control panel), lie down on the spinal disc decompression device 10, secure himself or herself to the spinal disc decompression device (e.g., using the strap members 128), and activate the treatment protocol via the controller. Additionally, the controller 18 can include at least one speaker (not shown) for providing auditory signals and/or instructions to the patient.

It will also be appreciated that the controller 18 can additionally or optionally be configured to enable remote monitoring and/or data storage capabilities. For example, the controller 18 can include a communications interface (not shown) for transmitting and/or receiving data between the controller and at least one remote device (not shown). Remote devices can include any device capable of connecting to and communicating with the controller 18. Examples of remote devices can include, but are not limited to, desktop computers, mp3 players, mobile phones, PDAs, game consoles, and set-top boxes. Various wire-based protocols, such as USB, Ethernet, and FireWire, and wireless protocols, such as Bluetooth and Wi-Fi, can be used to facilitate communication between the controller 18 and a remote device. In addition, it will be appreciated that various proprietary protocols can be developed for communicating between the controller 18 and a remote device.

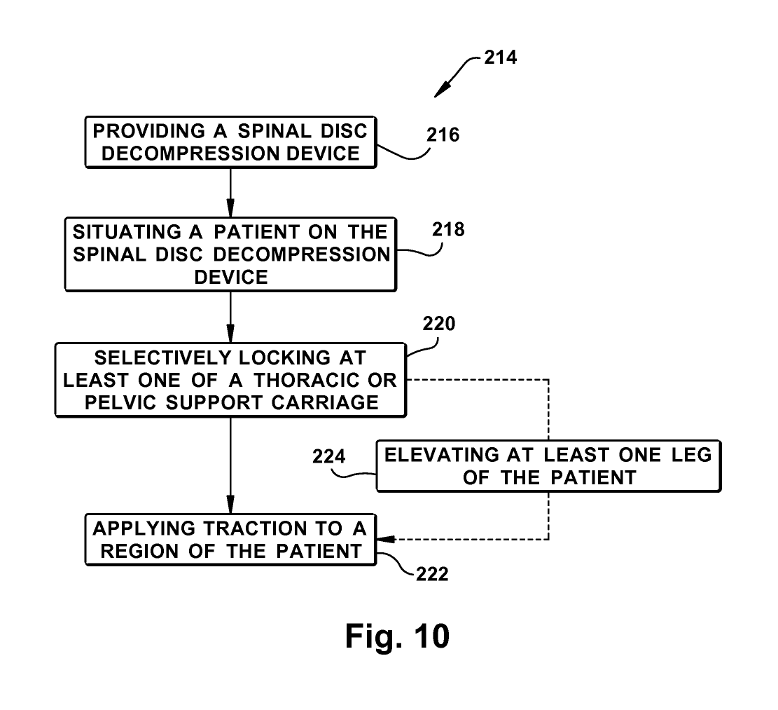

Another aspect of the present invention includes a method 214 (FIG. 10) for spinal disc decompression in the home of a patient. Unlike conventional spinal traction protocols, which require multiple trips to the doctor's office and do not provide treatment at night, the method 214 of the present invention allows for lower back traction (spinal disc decompression) separately, or in combination with, thoracic and cervical traction in a home-based setting during periods of wakefulness and sleep. The functional concept of the method 214 is to apply traction to a spinal structure (or structures) over an extended period of time (e.g., during nighttime or periods of sleep) and at a lower force than conventional spinal traction devices. In addition, patients in need of significant amounts of treatment may repeat the method numerous times (e.g., during the day) to speed the healing process within an injured spinal disc (or discs). Advantageously, the method 214 of the present invention offers a much more efficient use of time and provides a more thorough treatment protocol.

The method 214 includes providing a spinal disc decompression device 10 at Step 216. The spinal disc decompression device 10 can generally comprise a collapsible base unit 12, a traction mechanism 14, a series of patient support members 16, and a controller 18 (as described above). The dimensions and configuration of the spinal disc decompression device 10 will depend upon the height and weight of the patient being treated, as well as the particular back ailment(s) from which the patient is suffering. To accommodate a taller patient, for example, at least one extension member 82 can be included as part of the spinal disc decompression device 10. Additionally, it will be appreciated that the first and second leg support members 40 and 42 can be motorized or non-motorized depending upon the particular needs of the patient.

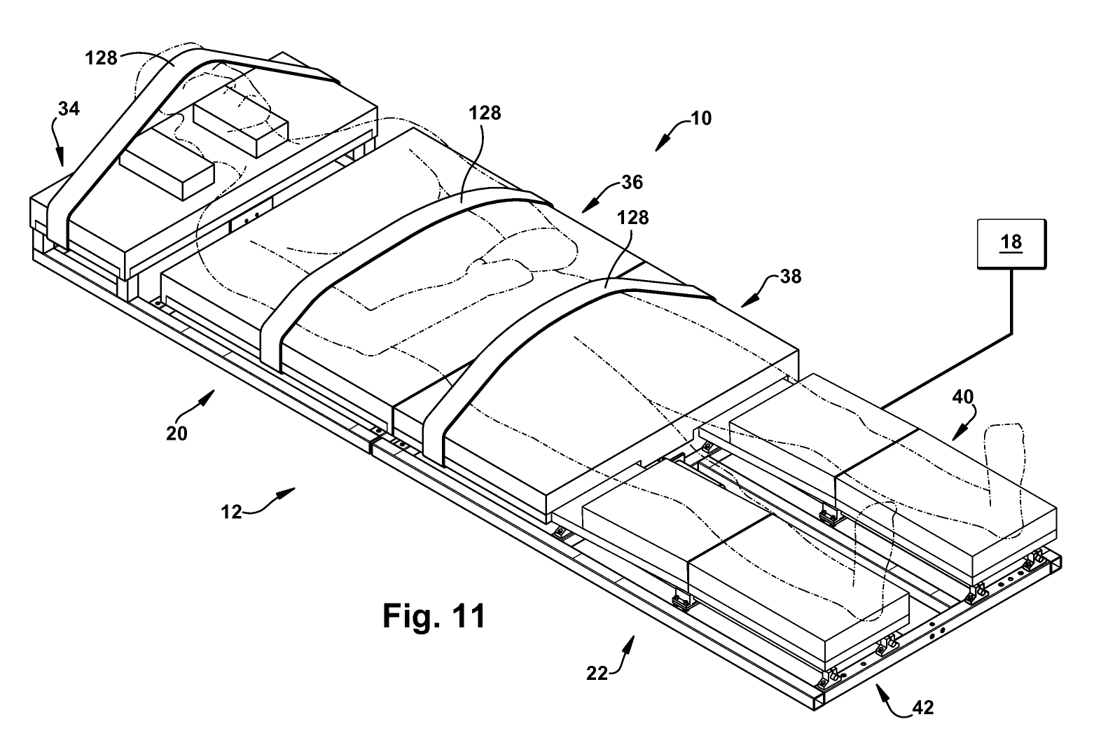

At Step 218, the patient is appropriately situated on the spinal disc decompression device 10. Depending upon the particular back ailment for which treatment is sought, the patient can be situated in a supine, prone, right or left side up position so that the patient's head, chest, and pelvis contact the cervical support member 34, the thoracic support carriage 36, and the pelvic support carriage 38 (respectively). Additionally, the patient can be situated on the spinal disc decompression device 10 so that the left and right legs of the patient are in contact with the first and second leg support members 40 and 42. As shown in FIG. 11, at least one strap member 128 can be placed over each of the patient's head, chest, and waist to secure the patient to the spinal disc decompression device 10. Although not shown, it will be appreciated that a strap member 128 (or multiple strap members) can also be placed over one or both of the patient's legs.

After the patient is secured to the spinal disc decompression device 10, at least one of the locking mechanisms 44 is selectively engaged to lock the thoracic support carriage 36 and/or the pelvic support carriage 38 in place (Step 220). As further exemplified below, the determination of which of the locking mechanisms 44 to engage will depend upon the particular back ailment(s) from which the patient is suffering. For example: (1) to prepare the spinal disc decompression device 10 for treatment of pelvic or lumbar back pain, a first locking mechanism 44′ is selectively engaged by bringing the first electromagnetic member 186′ into sufficient proximity with the first angle iron plate 190 to magnetically connect the first electromagnetic member with the first angle iron plate and thereby immobilize the thoracic support carriage 36; (2) to prepare the spinal disc decompression device for treatment of cervical spinal pain, a second locking mechanism 44″ is selectively engaged by bringing the second electromagnetic member 186″ into sufficient proximity with the fourth angle iron plate 200 to magnetically connect the second electromagnetic member and the fourth angle iron plate and thereby operably join the thoracic and pelvic support carriages 38; and (3) to prepare the spinal disc decompression device for full spine treatment, the first and second electromagnetic members are disengaged from the first and fourth angle iron plates (respectively) to disconnect the thoracic support carriage from the pelvic support carriage and thereby allow the thoracic support carriage to move in a foot ward direction. It will be appreciated that Step 220 of the method can be performed using tactile force, manually (e.g., using the controller 18), automatically (e.g., using a preprogrammed memory card), or a combination thereof.

At Step 222, the controller 18 is operated to apply traction to at least one of a cervical, thoracic, or thoracic/lumbar region of the patient. As discussed above, the controller 18 can be operated either manually or via a set of preprogrammed instructions (e.g., a memory card). During manual operation, for example, the patient or a medical practitioner can control the amount and duration of traction that is applied to the patient. Alternatively, during automatic operation, one or more signals encoding a series of preprogrammed instructions for applying traction to the patient can be delivered to the controller 18.

Unlike conventional traction devices and related therapies, the spinal disc decompression device 10 of the present invention applies intermittent traction to one or more bodily regions of a patient. Application of intermittent traction can be accomplished by applying traction to one or more bodily regions of the patient as follows: (1) applying traction at a first poundage; (2) changing the traction to a second poundage that is greater than the first poundage; (3) maintaining the second poundage for a period of time; and (4) changing the traction from the second poundage to the first poundage. This series of steps defines the intermittent application of traction and can be repeated as needed to bring pain relief to the patient. The particular poundage(s) and period(s) of time over which traction is applied can be varied as required. Additionally, the period(s) of time over which intermittent traction is applied can range from less than about 30 minutes to about 60 minutes, about 90 minutes, or even greater.

In one example of the present invention, cervical traction can be applied as followed: (1) start traction at about 10-15 pounds (e.g., 12 pounds); (2) gradually increase cervical traction to about 25-35 pounds (e.g., 30 pounds); (3) maintaining the traction for a a period of time (e.g., several minutes); (4) decreasing the traction to about 15-25 pounds (e.g., 20 pounds) for a period of time (e.g., several minutes) (rest period); (5) increasing traction to about 25-35 pounds (e.g., 30 pounds); and (6) repeating steps (1)-(5) as needed or as required by the treatment protocol.

In another example of the present invention, lumbar traction can be applied as follows: (1) start traction at about 25-35 pounds (e.g., 30 pounds); (2) gradually increase lumbar traction to about 85-95 pounds (e.g., 90 pounds); (3) maintain the traction for a period of time (e.g., several minutes); (4) decrease the traction to about 55-65 pounds (e.g., 60 pounds); (5) maintain the traction for a period of time (e.g., several minutes); (6) increase the traction to about 85-95 pounds (e.g., 90 pounds); and (7) repeating steps (1)-(6) as needed or required by the treatment protocol.

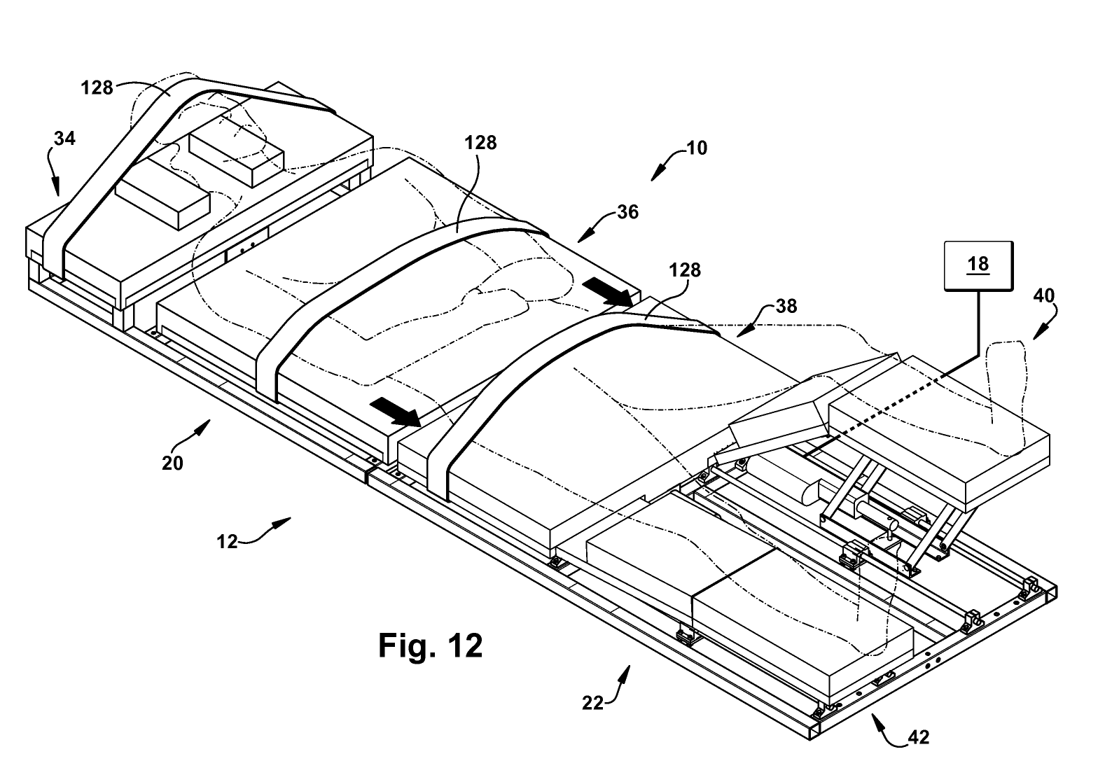

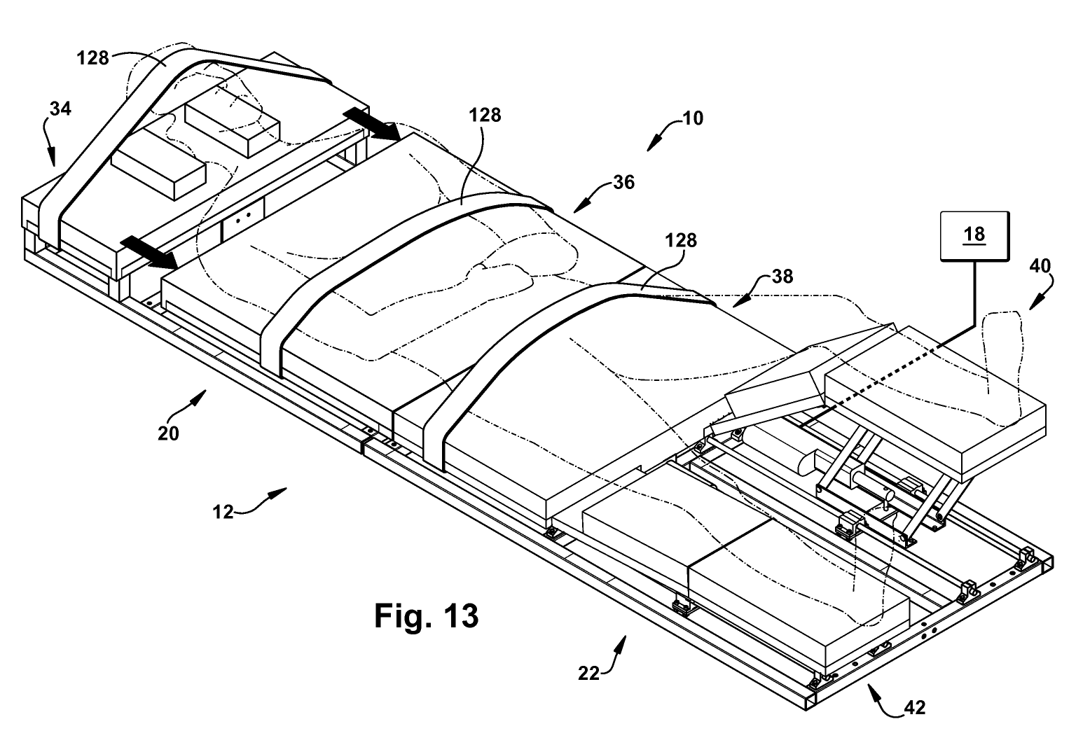

As noted above, the linear actuator 32 of the spinal disc decompression device 10 provides a linear force to the pelvic support carriage 38 and/or the thoracic support carriage 36 via selective engagement of the locking mechanisms 44. To apply traction to the lumbar region of a patient's back, for example, the thoracic support carriage 36 is first immobilized (as described above). The controller 18 then sends at least one signal to the linear actuator 32, which applies a linear force (via the drive rod 110) to the pelvic support carriage 38. As shown in FIG. 12, application of a linear force by the linear actuator 32 causes the pelvic support carriage 38 to move in a foot ward direction and thereby apply traction to the lumbar region of the patient. Alternatively, to apply traction to the cervical spine of a patient, the second locking mechanism 44″ is engaged to operably join the pelvic and thoracic support carriages 38 and 36 (as described above). The controller 18 then sends a signal to the linear actuator 32, which applies a linear force (via the drive rod 110) to the thoracic and pelvic support carriages 36 and 38. As shown in FIG. 13, application of the linear force causes the joined pelvic and thoracic support carriages 38 and 36 to move in a foot ward direction and thereby apply traction to the cervical spine of the patient.

It will be appreciated that the method 214 optionally includes activating at least one of the first and second leg support members 40 and 42 to elevate one or both of the patient's legs and thereby relieve sciatic pain (Step 224). The first leg support member 40 and/or the second leg support member 42 can be elevated prior to, during, or subsequent to Step 220. Additionally or optionally, the first leg support member 40 and/or the second leg support member 42 can be elevated during Step 222. The first and second leg support members 40 and 42 can be elevated so that the patient's thigh(s) is/are at an angle of about 1° to about 90° relative to the first surface 24 of the base unit 12. The first and second leg support members 40 and 42 can be operated independently or in tandem.

In one example of the method 214, a patient may visit his or her doctor complaining of lower back pain, severe radiating left leg pain and numb/tingling toes on the patient's left foot. After ruling out a pathological etiology, the doctor identifies lumbar disc herniation or bulge as the patient's tentative diagnosis. In addition to prescribing a steroidal anti-inflammatory medication, the doctor prescribes the home-based use of the spinal disc decompression device 10 for a period of about 2 to 4 weeks. Initially, the doctor recommends that the patient use the spinal disc decompression device 10 several times per day and, if needed, at nighttime while sleeping. The doctor gives the patient a pre-programmed memory card containing the prescribed traction protocol(s) and sends the patient home. The patient then arranges for the spinal disc decompression device 10 to be delivered to his or her home (e.g., via a local medical equipment supplier).

Once the spinal disc decompression device 10 is delivered to the patient's home, the patient secures himself/herself thereto (as described above). Next, the patient inserts the memory card into the controller 18 and initiates the treatment protocol(s) (e.g., by pressing “GO” on the controller). The spinal disc decompression device 10 automatically activates the first locking mechanism 44′ to immobilize the thoracic support carriage 36 and anchor the patient's upper body. The linear actuator 32 is then activated to cause the pelvic support carriage 38 to move in a foot ward direction. Since the patient's left leg is aching, the patient can simultaneously elevate his or her left leg by manipulating the appropriate button on the controller 18 and thereby cause the first leg support member 40 to flex and elevate the patient's leg to a desired position. Next, the patient can relax as treatment (i.e., intermittent traction) is applied gently to the patient's lower back for a desired period of time. When the patient is well again, the patient can contact the local medical equipment supplier to come and pick-up the spinal disc decompression device 10.

In another example of the method, a patient may have a herniated cervical disc and severe arm pain. In this case, the patient can lie down on the spinal disc decompression device 10 and place a strap member 128 comfortably around his or her head to firmly anchor the patient's head to the cervical support member 34. Upon pressing “GO” on the controller 18, the first locking mechanism 44′ can be disengaged to release the thoracic support member 36 and allow the second locking mechanism 44″ to operably join the pelvic support carriage 38 and the thoracic support carriage. The linear actuator 32 can then be activated to apply a linear force to the pelvic support carriage 38 (and thus the thoracic support carriage 36) so that the thoracic and pelvic support carriages move in a foot ward direction. This can create independent cervical traction. Alternatively, only the thoracic support carriage 36 may move in the foot ward direction and thereby produce substantially full spine traction (including the cervical spine). It will be appreciated that the cervical support member 34 can include a chin strap (not shown) to secure the patient's head during cervical traction. In this case, the cervical support member 34 can be split in the middle (as shown in FIG. 7A) to allow the patient to lie face down comfortably.

The method 214 of the present invention presents several advantages over conventional spinal traction therapies including, but not limited to: providing treatment in the comfort of a patient's home; the ability to self-administer or automatically apply therapy according to the individualized prescription of a medical practitioner; application of treatment during periods of sleep, which can substantially decrease nighttime pain and loss of sleep; decreased healing time for disc-related back pain; application of reduced traction forces, which can decrease the chance of injury and other unwanted side effects; avoidance or reduced dependence upon potentially-addictive pain medications; and avoiding the need for spinal injections and surgery.

From the above description of the invention, those skilled in the art will perceive improvements, changes and modifications. For example, the spinal disc decompression device 10 can include a feedback system (not shown) that prevents abnormally high traction when the cervical or full spine is being treated. Such a feedback system can be operably integrated with the locking mechanisms 44 so that built-in electronic controls can limit the amount of traction when the thoracic support carriage 36 is unlocked (e.g., for full spine traction) to prevent injury to the cervical spine. Alternatively or additionally, a connector (not shown) could be integrated into a strap member 128 intended to restrain a patient's head and provide feedback as to when the patient's head is safely anchored. It will be appreciated that the controller 18 may also include a “STOP” button that a patient or a medical practitioner can manipulate to immediately stop all traction. Such improvements, changes, and modifications are within the skill of the art and are intended to be covered by the appended claims.

Having described the invention, the following is claimed:

1. A portable spinal disc decompression device for use in a home setting, said spinal disc decompression device comprising:
a collapsible base unit including an upper base member and a lower base member, each of said upper and lower base members having oppositely disposed first and second surfaces;
a traction mechanism securely connected to said first surface of each of said upper and lower base members, said traction mechanism comprising a first plurality of linear bearings that is slidably connected to said upper base member, a second plurality of linear bearings that is slidably connected to said lower base member, and a linear actuator attached to said base unit;
a series of patient support members connected to said base unit, said series of patient support members including a cervical support member that is connected to said upper base member, a thoracic support carriage that is slidably mounted to said first plurality of linear bearings, a pelvic support carriage that is slidably mounted to said second plurality of linear bearings, and first and second leg support members connected to said lower base member, said series of patient of patient support members including a plurality of locking mechanisms for allowing selective linear movement of said thoracic and pelvic support carriages; and
a controller in electrical communication with said linear actuator, said controller for communicating a signal to said linear actuator to cause said linear actuator to apply a force to at least one of said thoracic and pelvic support carriages.

2. The spinal disc decompression device of claim 1, further comprising an extension member securely disposed between said upper and lower base members.

3. The spinal disc decompression device of claim 1, wherein at least one of said series of patient support members includes a recessed portion adapted to receive an ice pack or gel pack.

4. The spinal disc decompression device of claim 1, wherein each of said series of patient support members is made of a non-slip material.

5. The spinal disc decompression device of claim 1, wherein said plurality of locking mechanisms further comprises first and second electromagnetic members securely attached to first and second ends of said pelvic support carriage, respectively.

6. The spinal disc decompression device of claim 1, wherein each of said first and second leg support members comprises upper and lower leg pads respectively mounted upon upper and lower leg plates, said upper and lower leg plates being hingedly connected to one another and each of said upper leg plates being hingedly connected to said pelvic support carriage, each of said lower leg plates being connected to a lift mechanism for selectively elevating said first and second leg support members.

7. A portable spinal disc decompression device for use in a home setting, said spinal disc decompression device comprising:
a collapsible base unit including an upper base member and a lower base member, each of said upper and lower base members having oppositely disposed first and second surfaces;
a traction mechanism securely connected to said first surface of each of said upper and lower base members, said traction mechanism comprising a first plurality of linear bearings that is slidably connected to said upper base member, a second plurality of linear bearings that is slidably connected to said lower base member, and a linear actuator attached to said base unit;
a series of patient support members connected to said base unit, said series of patient support members including a cervical support member that is connected to said upper base member, a thoracic support carriage that is slidably mounted to said first plurality of linear bearings, a pelvic support carriage that is slidably mounted to said second plurality of linear bearings, and first and second leg support members connected to said lower base member, each of said first and second leg support members comprising upper and lower leg pads respectively mounted upon upper and lower leg plates, said upper and lower leg plates being hingedly connected to one another and each of said upper leg plates being hingedly connected to said pelvic support carriage, each of said lower leg plates being connected to a lift mechanism for selectively elevating said leg support members relative to said lower base member; and
a controller in electrical communication with said linear actuator, said controller for communicating a signal to said linear actuator to cause said linear actuator to apply a force to at least one of said thoracic and pelvic support carriages.

8. The spinal disc decompression device of claim 7, further comprising an extension member securely disposed between said upper and lower base members.

9. The spinal disc decompression device of claim 7, wherein at least one of said series of patient support members includes a recessed portion adapted to receive an ice pack or gel pack.

10. The spinal disc decompression device of claim 7, wherein each of said series of patient support members is made of a non-slip material.

11. The spinal disc decompression device of claim 7, wherein said series of patient of patient support members further includes a plurality of locking mechanisms for allowing selective linear movement of said thoracic and pelvic support carriages.

12. The spinal disc decompression device of claim 11, wherein said plurality of locking mechanisms further comprises first and second electromagnetic members securely attached to first and second ends of said pelvic support carriage, respectively.

13. A method for spinal disc decompression in the home of a patient, said method comprising the steps of:
providing a portable spinal disc decompression device comprising a collapsible base unit, a traction mechanism, a series of patient support members, and a controller, the base unit including an upper base member and a lower base member, each of the upper and lower base members having oppositely disposed first and second surfaces, the traction mechanism being securely connected to the first surface of each of the upper and lower base members, the traction mechanism comprising a first plurality of linear bearings that is slidably connected to the upper base member, a second plurality of linear bearings that is slidably connected to the lower base member, and a linear actuator attached to the base unit, the series of patient support members being connected to the base unit and including a cervical support member that is connected to the upper base member, a thoracic support carriage that is slidably mounted to the first plurality of linear bearings, a pelvic support carriage that is slidably mounted to the second plurality of linear bearings, and first and second leg support members connected to the lower base member, the series of patient support members including a plurality of locking mechanisms, the controller being in electrical communication with the linear actuator;
situating the patient on the spinal disc decompression device so that the head, chest, waist, and legs of the patient respectively engage the cervical support member, the thoracic support carriage, the pelvic support carriage, and the first and second leg support members;
selectively engaging at least one of the locking mechanisms to lock at least one of the thoracic or pelvic support carriages in place;
optionally elevating at least one of the first or second leg support members; and
operating the controller so that traction is applied to at least one of a cervical, thoracic, or thoracic/lumbar region of the patient.

14. The method of claim 14, wherein traction is applied intermittently to the patient.

15. The method of claim 14, wherein application of the intermittent traction comprises the following steps:
(a) applying traction to the patient at a first poundage;
(b) changing the traction to a second poundage that is greater than the first poundage;
(c) maintaining the second poundage for a period of time;
(d) changing the traction from the second poundage to the first poundage; and
(e) optionally repeating steps (a)-(d).

16. The method of claim 14, wherein traction is applied to the patient while the patient is sleeping.

17. The method of claim 14, wherein said step of operating the controller further includes the step of delivering a signal to the controller, the signal encoding a series of preprogrammed instructions for applying intermittent traction to the patient.

18. The method of claim 14, further comprising the steps of:
engaging a first locking mechanism to immobilize the thoracic support carriage; and
operating the controller to deliver a force to the pelvic support carriage and thereby apply traction to a pelvic region of the patient.

19. The method of claim 14, further comprising the steps of:
engaging a second locking mechanism to operably join the thoracic support carriage and pelvic support carriage; and
operating the controller to deliver a force to the pelvic and thoracic support carriages and thereby apply traction to a cervical spine region of the patient.

20. The method of claim 14, further comprising the steps of:
disengaging first and second locking mechanisms to unlock the thoracic support carriage; and
operating the controller to deliver a force to the thoracic support carriage and thereby apply traction to substantially the entire spine of the patient.
